(19) United States
(12) Patent Application Publication
(10) Pub. No.: US 2022/0251588 A1
(43) Pub. Date: Aug. 11, 2022

(54) COMPOSITIONS AND METHODS FOR CHROMOSOME REARRANGEMENT

(71) Applicant: Monsanto Technology LLC, St. Louis, MO (US)

(72) Inventors: Charles Lester Armstrong, St. Louis, MO (US); Michelle Lee Gasper, St. Charles, MO (US); Andrei Y. Kouranov, Chesterfield, MO (US); Richard J. Lawrence, Kirkwood, MO (US); Samuel Sukhwan Yang, Ballwin, MO (US)

(21) Appl. No.: 17/630,465

(22) PCT Filed: Aug. 4, 2020
(86) PCT No.: PCT/US2020/044900

§ 371 Date: Jan. 26, 2022

Related U.S. Application Data
(60) Provisional application No. 62/882,854, filed on Aug. 5, 2019.

(51) Int. Cl. C12N 15/82; C12Q 1/6876

(52) U.S. Cl. CPC C12N 15/8257; C12N 2830/42; C12Q 2600/158; C12Q 1/6876

(57) ABSTRACT

Methods and compositions for evaluating the efficiency of chromosomal rearrangement are provided. In some examples, systems comprising a first DNA molecule comprising the N-terminal portion of a first split reporter coding sequence linked to the C-terminal portion of a second split reporter coding sequence via a first intron, and a second DNA molecule comprising the N-terminal portion of said second split reporter coding sequence linked to the C-terminal portion of said first split reporter coding sequence via a second intron. The introns comprise at least one target site recognized by a genome editing reagent, such as a recombinase or endonuclease, such that recombination results in expression of the first or second reporter coding sequence following splicing of the introns.

### REFERENCE TO RELATED APPLICATION

This application claims the benefit of U.S. Provisional Application No. 62/882,854, filed Aug. 5, 2019, which is herein incorporated by reference in its entirety.

### FIELD OF THE INVENTION

The present invention relates to the field of agricultural biotechnology, and more specifically to constructs and methods for evaluating chromosomal rearrangements in plant cells.

### INCORPORATION OF SEQUENCE LISTING

A sequence listing contained in the file named “MONS449WO_ST25.txt” which is 36.7 kilobytes (measured in MS-Windows®) and created on Aug. 4, 2020, comprises 48 nucleotide sequences, is filed electronically herewith and incorporated by reference in its entirety.

### BACKGROUND

Recombination at a desired locus has the potential to allow for movement of DNA containing valuable genetic loci into commercial germlines, which could be of enormous value for crop improvement. Although methods exist for modifying plant genomes using cis or trans chromosomal rearrangement, these previously known methods rely primarily on genetic selection to identify modifications to plant genomes. Existing methods are therefore inefficient and expensive due to the considerable effort required to produce and identify plants comprising desired genome modifications. Improved methods for evaluating the efficiency of cis or trans chromosomal rearrangement and identifying advantageous genome modifications are therefore needed.

### SUMMARY

In a first aspect, a pair of recombinant DNA molecules is provided, comprising: a) a first DNA molecule comprising an N-terminal portion of a first reporter coding sequence and a C-terminal portion of a second reporter coding sequence that flank a first intron, wherein said first intron comprises a first target site recognizable by a first recombinase or endonuclease; and b) a second DNA molecule comprising an N-terminal portion of said second reporter coding sequence and a C-terminal portion of said first reporter coding sequence that flank a second intron, wherein said second intron comprises a second target site recognizable by a second recombinase or endonuclease. Following recombination between said first and second DNA molecules at said target sites, the N-terminal and C-terminal portions of said first reporter coding sequence form an expression cassette capable of expressing said first reporter coding sequence, and the N-terminal and C-terminal portions of said second reporter coding sequence form an expression cassette capable of expressing said second reporter coding sequence. Said first or said second reporter coding sequence may encode a fluorescent marker, an enzymatic marker, or an herbicide tolerance selection marker, for example green fluorescent protein (GFP), β-glucuronidase (GUS), or CP4. Said recombinase may be selected from the group consisting of a Cre recombinase, a FLP recombinase, and a TALE recombinase (TALER). For example, said recombinase may be a Cre recombinase, and said target site may be a Lox site. Said endonuclease may be selected from the group consisting of a meganuclease, a Zinc Finger nuclease, a TALEN and a CRISPR-associated (Cas) endonuclease. For example, said endonuclease may be a Cas9 or Cpf2 endonuclease. Said first DNA molecule may further comprise a sequence encoding a Cas protein, and said second DNA molecule may further comprise a sequence encoding a guide RNA. Alternatively, said first DNA molecule may further comprise a sequence encoding a guide RNA, and said second DNA molecule may further comprise a sequence encoding a Cas protein. Expression of said sequence encoding a recombinase or endonuclease may be driven by a constitutive promoter, a tissue-specific promoter, or a meiotic promoter. For example, said promoter may be selected from the group consisting of an At EASE promoter, an At DMC1 promoter, a ubiquitous promoter 1, a rice actin promoter, or a soy BURP09 promoter.

In another aspect, a plant cell comprising a pair of recombinant DNA molecules described herein is provided. Transgenic plants, plant seeds, or plant parts comprising a pair of recombinant DNA molecules described herein are further provided.

In a further aspect, methods for detecting recombination in a cis or trans chromosomal rearrangement system are provided, comprising: a) obtaining a transgenic plant transformed with a first DNA molecule comprising an N-terminal portion of a first reporter coding sequence and a C-terminal portion of a second reporter coding sequence that flank a first intron; b) obtaining a transgenic plant transformed with a second DNA molecule comprising an N-terminal portion of said second reporter coding sequence and a C-terminal portion of said first reporter coding sequence that flank a second intron; c) crossing said first transgenic plant with said second transgenic plant to produce a progeny plant comprising said first DNA molecule and said second DNA molecule; d) providing to at least a first cell of said progeny plant or a progeny thereof comprising said first DNA molecule and said second DNA molecule a recombinase or endonuclease that recognizes a target site in said first intron or a target site in said second intron; and e) detecting recombination between said first and second DNA molecules at said target sites based on the expression of said first and second reporter coding sequences. In some embodiments, said first DNA molecule further comprises a sequence encoding a Cas protein, and said second DNA molecule further comprises a sequence encoding a guide RNA. Alternatively, said first DNA molecule further comprises a sequence encoding a guide RNA, and said second DNA molecule further comprises a sequence encoding a Cas protein. Said first or said second reporter coding sequence may encode a fluorescent marker, an enzymatic marker, or an herbicide tolerance selection marker. Said first or said second reporter coding sequence may encode GFP, GUS, or CP4. Said recombinase may be selected from the group consisting of a Cre recombinase, a FLP recombinase, and a TALE recombinase (TALER). Said endonuclease is selected from the group consisting of a CRISPR-associated (Cas) endonuclease or a Cfp1 endonuclease.

In another aspect, methods for detecting recombination in a cis or trans chromosomal rearrangement system are provided, comprising: a) obtaining a transgenic plant comprising: i) a first DNA molecule comprising an N-terminal portion of a first reporter coding sequence and a C-terminal portion of a second reporter coding sequence that flank a first intron, wherein said first intron comprises a first target site recognizable by a first recombinase or endonuclease; and ii) a second DNA molecule comprising an N-terminal portion of said second reporter coding sequence and a C-terminal portion of said first reporter coding sequence that flank a second intron, wherein said second intron comprises a second target site recognizable by a second recombinase or endonuclease; and wherein said first DNA molecule or said second DNA molecule further comprises a sequence encoding said first or said second recombinase or endonuclease; b) detecting recombination between said first and second DNA molecules at said target sites based on the expression of said first and second reporter coding sequences. Said first or said second reporter coding sequence may encode a fluorescent marker, an enzymatic marker, or an herbicide tolerance selection marker. Said first or said second reporter coding sequence may encode GFP, GUS, or CP4. Said recombinase may be selected from the group consisting of a Cre recombinase, a FLP recombinase, and a TALER. Said endonuclease may be selected from the group consisting of a Cas endonuclease or a Cfp1 endonuclease.

### BRIEF DESCRIPTION OF THE DRAWINGS

FIG. 1 shows a schematic representation of a construct useful for testing the efficiency of recombination in cells. This construct comprises a CaMV promoter, an N-terminal portion of a GFP coding sequence, an intron comprising at least one LoxP site, a target site for a CRISPR-associated protein, and a C-terminal portion of a CP4 coding sequence.

FIG. 2 shows a schematic representation of a construct for use in combination with the construct shown in FIG. 1. The second construct comprises a ubiquitous promoter 1, an N-terminal portion of the CP4 coding sequence, an intron comprising at least one LoxP site, a gRNA target site, and a C-terminal portion of the GFP coding sequence.

FIG. 3 shows a schematic representation of a set of constructs (Vector A and Vector B) designed for detecting and optimizing recombination in a cis or trans chromosomal rearrangement system as described herein. Vector A comprises a CaMV promoter, an N-terminal portion of a GFP coding sequence, an intron comprising a target site recognized by a genome editing reagent, such as a recombinase or endonuclease, and a C-terminal portion of a CP4 coding sequence. Vector B comprises a ubiquitous promoter 1, an N-terminal portion of the CP4 coding sequence, an intron comprising a target site recognized by a genome editing reagent, such as a recombinase or endonuclease, a gRNA target site, and a C-terminal portion of the GFP coding sequence. Either or both of these constructs may be transformed into a plant using standard plant transformation methods.

FIG. 4 shows a schematic diagram of plasmid recombination according to the disclosed method and induced by expression of editing reagents (Cre or Cas9).

FIG. 5 shows recombination efficiency measured as a percentage of GFP-expressing cells in corn protoplasts using the disclosed system.

FIG. 6 shows a schematic of constructs for a Cre split reporter system for determining recombination efficiency in soy cotyledon protoplasts. Vector A comprises a split reporter gene linked by an intron comprising Lox and gRNA target sequences with or without a further Cre coding sequence driven by a separate promoter. Vector B comprises the intron, Lox, and gRNA target sequences that are in Vector A. Vector C is a positive control.

FIG. 7 shows the expected products of recombination when Vectors A, B, and C of FIG. 7 are introduced into cells.

FIG. 8 shows recombination efficiency measured as a percentage of GFP-expressing cells in soy protoplasts using the constructs diagrammed in FIG. 7.

FIG. 9 shows a schematic diagram of constructs for a Cpf1 split reporter system for determining recombination efficiency in soy cotyledon protoplasts. Vector A comprises a split reporter gene linked by an intron comprising Lox and gRNA target sequences with or without a further Cpf1 coding sequence driven by a separate promoter. Vector B comprises the intron, Lox, and gRNA target sequences that are in Vector A. Vector C is a positive control.

FIG. 10 shows recombination efficiency measured as a percentage of GFP-expressing cells in soy protoplasts using the constructs diagrammed in FIG. 10.

FIG. 11 shows a schematic of chromosomal rearrangements in R1 homozygous seeds harvested from corn plants comprising a split reporter system as disclosed.

### DETAILED DESCRIPTION

Recombination at specific loci can be extremely useful for moving DNA containing valuable genetic material into a recipient plant line. However, detection of cis or trans chromosomal rearrangement has previously been carried out using costly and labor-intensive genetic selection methods. The instant disclosure provides improved methods for evaluating the efficiency of cis or trans chromosomal rearrangement and identifying advantageous genome modifications.

The shortcomings of previous systems for evaluation of chromosome rearrangement are compounded by the fact that they have been focused on the use of single genome editing reagents, and do not enable the evaluation and comparison of multiple genome editing reagents simultaneously. Assessment of genome edits has also conventionally been aimed at detection of small molecular changes, and efficient systems have not been developed for evaluation of chromosome modifications such as cis and trans location of chromosomes.

In order to address these limitations, the present disclosure provides an efficient and cost-effective system for identifying genome edits in cells. In certain embodiments, a system as disclosed herein provides a first DNA molecule comprising the N-terminal portion of a first split reporter coding sequence linked to the C-terminal portion of a second split reporter coding sequence via a first intron. In one embodiment, the intron comprises at least one target site recognized by a genome editing reagent, such as a LoxP site or a gRNA target site. A second DNA molecule comprises the N-terminal portion of the second split reporter coding sequence linked to the C-terminal portion of the first split reporter coding sequence via a second intron, and the second intron also comprises at least one target site recognized by a genome editing reagent, such as a LoxP site or a gRNA target site. Recombination results in the N-terminal and the C-terminal portions of the first reporter coding sequence being operably linked via the first intron, and the N-terminal and the C-terminal portions of the second reporter coding sequence being operably linked via the second intron. The resulting sequences are transcribed and processed to remove the introns, and one or both of the reporter coding sequences is expressed such that it can be detected.

The disclosed systems represent a significant advantage in the art because they allow for the rapid and non-destructive assessment of genome editing using fluorescent, enzymatic, or herbicide tolerance markers. If an exchange has occurred either in cis or trans, the marker is expressed and edits can be measured. The use of herbicide tolerance markers in the disclosed systems further allows for rapid selection of edited genomes.

The systems described herein also allow determination of the frequency of chromosome rearrangements in cis and in trans, as well as the evaluation of multiple genome editing reagents simultaneously. The efficiency of genome editing reagents driven by various promoters can also be tested. Using the disclosed system, the frequency and transmissibility of genome edits resulting from genome editing reagents under control of various regulatory elements can be compared to optimize gene editing in plant cells.

### I. Constructs for Detecting and Optimizing Chromosomal Rearrangement

To allow for efficient detection of chromosomal rearrangement, provided herein are methods and constructs comprising a first and a second split reporter gene coding sequence. As used herein, term “split reporter” or “split reporter coding sequence” refers to a reporter gene wherein the N-terminal portion of the reporter gene coding sequence is not operably linked to the C-terminal portion of the reporter gene coding sequence. A recombination event can operably link the N-terminal portion of a split reporter to the C-terminal portion of a split reporter, resulting in a sequence capable of expressing the reporter gene.

In several embodiments, a pair of recombinant DNA molecules is provided. A first DNA molecule may comprise an N-terminal portion of a first reporter coding sequence and a C-terminal portion of a second reporter coding sequence that flank a first intron, wherein said first intron comprises a first target site recognizable by a first recombinase or endonuclease. A second DNA molecule may comprise an N-terminal portion of said second reporter coding sequence and a C-terminal portion of said first reporter coding sequence that flank a second intron, wherein said second intron comprises a second target site recognizable by a second recombinase or endonuclease. When the first and second DNA molecules are located at specific chromosomal locations, recombination between those loci occurs, the N-terminal and C-terminal portions of the first and second reporter coding sequences are operably linked to form expression cassettes capable of expressing the first and second reporter coding sequences. The expression of a reporter coding sequence can therefore be used to determine recombination efficiency between the chromosomal locations where the DNA molecules are located. The construct and methods currently provided therefore allow for rapid and non-destructive assessment of genome editing, determination of the frequencies of chromosome rearrangements in cis and trans at different locations or between chromosomes, as well as methods of testing the efficiency of genome editing machinery driven by various promoters.

### Reporter Coding Sequences

Reporter coding sequences useful in the present invention include any detectable reporter molecules including fluorescent markers such as green fluorescent protein, enzymatic color markers, or herbicide tolerance selection markers. These include sequences encoding any type of detectable marker, such as fluorescent markers, enzymatic markers, or selectable markers. Commonly used selectable marker genes include markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known. Markers conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroA or EPSPS) are also useful in the disclosed systems. Examples of such selectable markers are illustrated in U.S. Pat. Nos. 5,550,318; 5,633,435; 5,780,708 and 6,118,047.

Split reporter coding sequences may be split at any point within the coding sequence, so long as the expression generated by the reconstituted N-terminus and C-terminus is detectable at a significantly higher level than either the N-terminus or C-terminus alone. For example, the N-terminus of a split reporter sequence may comprise at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% of the full-length reporter coding sequence. As described herein, the N-terminus of a split reporter sequence may be incorporated into a first DNA molecule at a first specific chromosomal location, while the C-terminus of a split reporter sequence may be incorporated into a second DNA molecule at a second specific chromosomal location, such that detection of the reconstituted reporter coding sequence indicates recombination between those two chromosomal locations.

### Introns

In several embodiments, a DNA construct provided herein comprises a first DNA molecule comprising an N-terminal portion of a first split reporter coding sequence linked to a C-terminal portion of a second split reporter coding sequence via a first intron. The intron comprises at least one target site recognized by a recombinase or endonuclease, such as a LoxP site or a gRNA target site. A second DNA molecule comprises the N-terminal portion of the second split reporter coding sequence linked to the C-terminal portion of the first split reporter coding sequence via a second intron. Recombination results in the N-terminal and the C-terminal portions of the first reporter coding sequence being linked via the first intron, and the N-terminal and the C-terminal portions of the second reporter coding sequence being linked via the second intron. The resulting sequences are transcribed and processed to remove the introns, reconstituting the full-length reporter sequences, so expression of the reporters can be detected.

### Genome Editing Reagents and Target Sites

DNA constructs described herein comprise intron sequences comprising one or more target sites for genome editing reagents. As used herein, a “target site” for genome editing reagent refers to a polynucleotide sequence that is bound and/or cleaved by a genome editing reagent such as an endonuclease or recombinase. A target site may comprise at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides of a sequence recognized by a genome editing reagent. A target site for an RNA-guided nuclease may comprise the sequence of either complementary strand of a double-stranded nucleic acid (DNA) molecule or chromosome at the target site.

A genome editing reagent may bind to a target site, such as via a non-coding guide nucleic acid (e.g., a CRISPR RNA (crRNA) or a single-guide RNA (sgRNA)). A targeter sequence of a guide nucleic acid may be complementary to a target site (e.g., complementary to either strand of a double-stranded nucleic acid molecule or chromosome at the target site). It will be appreciated that perfect identity or complementarity may not be required for a targeter sequence of a guide nucleic acid to bind or hybridize to a target site. For example, at least 1, at least 2, at least 3, at least 4, at least 5, at least 6, at least 7, or at least 8 mismatches (or more) between a target site and a targeter sequence of a guide nucleic acid may be tolerated. A “target site” also refers to the location of a polynucleotide sequence that is bound and cleaved by any other genome editing reagent that may not be guided by a guide nucleic acid molecule, such as a meganuclease, zinc finger nuclease (ZFN), a transcription activator-like effector nuclease (TALEN), etc., to introduce a double stranded break, single-stranded nick, or other modification into the polynucleotide sequence and/or its complementary DNA strand. In some embodiments, a “target site” refers to a recognition site for a recombinase, such a Lox or FRT site.

Target sites described herein may be recognized by any genome editing reagent, including recombinases and endonucleases, such as zinc-finger nucleases, engineered or native meganucleases, TALE-endonucleases, and RNA-guided endonucleases including Cas9, Cpf1, CasX, CasY, and other endonucleases used in CRISPR systems.

In several embodiments, DNA constructs comprise target sites recognized by CRISPR-associated nucleases (non-limiting examples of CRISPR associated nucleases include Cas1, Cas1B, Cas2, Cas3, Cas4, Cas5, Cas6, Cas7, Cas8, Cas9 (also known as Csn1 and Csx12), Cas10, Cpf1 (also known as Cas12a), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, CasZ , Mad7, homologs thereof, or modified versions thereof.

In some embodiments, DNA constructs comprise target sites recognized by a recombinase, such as a Cre recombinase, a Gin recombinase, a Flp recombinase, and a Tnp 1 recombinase. If the recombinase is a Cre recombinase, the target site may be a Lox site, such as a LoxP, Lox 2272, LoxN, Lox 511, Lox 5171, Lox71, Lox66, M2, M3, M7, or M11 site.

### Regulatory Elements

Constructs may further include regulatory elements that are functional in the host cell in which the construct is to be expressed. A person of ordinary skill in the art can select regulatory elements for use in bacterial host cells, yeast host cells, plant host cells, insect host cells, mammalian host cells, and human host cells. Regulatory elements include promoters, transcription termination sequences, translation termination sequences, enhancers, and polyadenylation elements. As used herein, the term “construct” or “expression construct” refers to a combination of nucleic acid sequences that provides for transcription of an operably linked nucleic acid sequence. As used herein, “operably linked” means two DNA molecules linked in manner so that one may affect the function of the other. Operably linked DNA molecules may be part of a single contiguous molecule and may or may not be adjacent. For example, a promoter is operably linked with a polypeptide-encoding DNA molecule in a DNA construct where the two DNA molecules are so arranged that the promoter may affect the expression of the DNA molecule.

As used herein, the term “heterologous” refers to the relationship between two or more items derived from different sources and thus not normally associated in nature. For example, a protein-coding recombinant DNA molecule is heterologous with respect to an operably linked promoter if such a combination is not normally found in nature. In addition, a particular recombinant DNA molecule may be heterologous with respect to a cell, seed, or organism into which it is inserted when it would not naturally occur in that particular cell, seed, or organism.

II. Methods for Detecting and Optimizing Chromosomal Rearrangement

Several embodiments relate to plant cells, plant tissues, plants, and seeds that comprise a construct as described herein. Plant cells, plant parts, and seeds may be transformed with a disclosed DNA construct by any method known in the art. Suitable methods for transformation of host plant cells are well known in the art, and include virtually any method by which DNA or RNA can be introduced into a cell (for example, where a recombinant DNA construct is stably integrated into a plant chromosome or where a recombinant DNA construct or an RNA is transiently provided to a plant cell). Two effective methods for cell transformation are Agrobacterium-mediated transformation and microprojectile bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. US 5,591,616, which is incorporated herein by reference in its entirety. Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as micro spores and pollen. Callus can be initiated from tissue sources including, but not limited to, immature or mature embryos, hypocotyls, seedling apical meristems, microspores and the like. Cells containing a transgenic nucleus are grown into transgenic plants. The regenerated plant can then be used to propagate additional plants.

In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or an herbicide. Any of the herbicides to which plants of this disclosure can be resistant is an agent for selective markers. Potentially transformed cells are exposed to the selective agent. In the population of surviving cells are those cells where, generally, the resistance-conferring gene is integrated and expressed at sufficient levels to permit cell survival. Cells can be tested further to confirm stable integration of the exogenous DNA. Further, the location of genetic material introduced into the genome of a plant cell can be determined by targeted sequencing.

### Recombinase or Endonuclease on Separate Construct

In several embodiments, constructs comprising a first split reporter and a second split reporter as described herein are transformed into plant cells, and plants are regenerated from the cells. The transgene location in the genome is determined, for example by targeted sequencing. Events comprising the first split reporter construct at a first specific chromosomal location and the second split reporter construct at a second specific location are identified. Plants comprising the first split reporter construct are crossed with plants comprising the second split reporter construct to produce F1 plants comprising both constructs. These F1 plants are transformed with a further construct encoding a genome editing reagent, such as a recombinase or endonuclease, for example Cas9, Cpf1, or Cre protein, corresponding to the target sites in the first and/or second split reporter construct. Recombination at the specific chromosomal locations where the split reporter constructs are located is evaluated by detecting expression of the reporter sequences.

### Recombinase or Endonuclease on Split Reporter Construct

In further embodiments, a first and/or second split reporter construct further comprises a sequence encoding a genome editing reagent, such as a recombinase or endonuclease, for example Cas9, Cpf1, or Cre protein, under the control of a promoter. The first and second split reporter constructs are transformed into plant cells, and plants are regenerated from the cells. The transgene location in the plant genome is determined, for example by targeted sequencing. Events comprising the first split reporter construct at a first specific chromosomal location and the second split reporter construct at a second specific location are identified. Plants comprising the first split reporter construct are crossed with plants comprising the second split reporter construct to produce F1 plants comprising both constructs. Recombination at the specific chromosomal locations where the split reporter constructs are located is evaluated by detecting expression of the reporter sequences.

### Guide RNA on Split Reporter Construct

In yet further embodiments, a first split reporter construct further comprises a sequence encoding a genome editing reagent, such as a an RNA-guided nuclease, for example Cas9or Cpf1 protein, under the control of a promoter. A second split reporter construct further comprises a sequence encoding a guide RNA (gRNA) directed to a target sequence within the intron of the first split reporter sequence. The first and second split reporter constructs are transformed into plant cells, and plants are regenerated from the cells. The transgene location in the plant genome is determined, for example by targeted sequencing. Events comprising the first split reporter construct at a first specific chromosomal location and the second split reporter construct at a second specific location are identified. Plants comprising the first split reporter construct are crossed with plants comprising the second split reporter construct to produce F1 plants comprising both constructs. Recombination at the specific chromosomal locations where the split reporter constructs are located is evaluated by detecting expression of the reporter sequences.

Several embodiments relate to plant cells, plant tissue, plant seed and plants produced by the methods disclosed herein. Plants may be monocots or dicots, and may include, for example, rice, wheat, barley, oats, rye, sorghum, maize, grapes, tomatoes, potatoes, lettuce, broccoli, cucumber, peanut, melon, leeks, onion, soybean, alfalfa, sunflower, cotton, canola, and sugar beet plants.

### III. Definitions

Unless defined otherwise herein, terms are to be understood according to conventional usage by those of ordinary skill in the relevant art. Examples of resources describing many of the terms related to molecular biology used herein can be found in Alberts et al., Molecular Biology of The Cell, 5th Edition, Garland Science Publishing, Inc.: New York, 2007; Rieger et al., Glossary of Genetics: Classical and Molecular, 5th edition, Springer-Verlag: New York, 1991; King et al, A Dictionary of Genetics, 6th ed., Oxford University Press: New York, 2002; and Lewin, Genes IX, Oxford University Press: New York, 2007. The nomenclature for DNA bases as set forth at 37 C.F.R. § 1.822 is used.

“Construct” or “DNA construct” or “expression construct” as used herein refers to a polynucleotide sequence comprising at least a first polynucleotide sequence operably linked to a second polynucleotide sequence.

“Donor molecule” or “donor DNA” or “template molecule” or “template DNA” or “donor DNA cassette” as used herein refers to a nucleic acid molecule which can serve as a template for modification of a genome, often at a specific location in the genome. In one example, a genome editing technique may involve disrupting the genome at a specific location (for example, using an endonuclease) and modifying the genome at that location based on the sequence of a donor molecule. A “donor DNA cassette” may comprise homology arms (HA) which are regions of the donor DNA cassette identical to the genomic regions flanking the 5′ and 3′ sides of the genomic site targeted for homologous integration. The donor DNA cassette may be configured with a 5′ homology arm operably linked to the donor DNA operably linked to a 3′ homology arm. In one example, the homology arms are the site of recombination resulting in the site-directed targeted integration of the donor DNA.

“Expression cassette” as used herein refers to a polynucleotide sequence comprising at least a first polynucleotide sequence capable of initiating transcription of an operably linked second polynucleotide sequence and optionally a transcription termination sequence operably linked to the second polynucleotide sequence.

“Genome editing” or “genome modification” as used herein refers to a process of modifying the genome of an organism, often at a specific location in the genome. Exemplary methods for introducing donor polynucleotides into a plant genome or modifying genomic DNA of a plant include the use of sequence-specific nucleases, such as zinc-finger nucleases, engineered or native meganucleases, TALE-endonucleases, or RNA-guided endonucleases, and examples include the use of CRISPR/Cas9, CRISPR/Cpf1, and Cre/Lox systems for the purpose of introducing a donor or template DNA sequence at a specific location in the genome.

“Guide molecule” or “guide RNA (gRNA)” as used herein refers to a nucleic acid molecule used to target at least one region of a genome for modification using genome editing techniques.

“Palindromic sequences” are nucleic acid sequences that are the same whether read 5′ to 3′ on one strand or 3′ to 5′ on the complementary strand with which it forms a double helix. A nucleotide sequence is the to be a palindrome if it is equal to its reverse complement. A palindromic sequence can form a hairpin.

“Percent identity” or “% identity” means the extent to which two optimally aligned DNA or protein segments are invariant throughout a window of alignment of components, for example nucleotide sequence or amino acid sequence. An “identity fraction” for aligned segments of a test sequence and a reference sequence is the number of identical components that are shared by sequences of the two aligned segments divided by the total number of sequence components in the reference segment over a window of alignment which is the smaller of the full test sequence or the full reference sequence.

“Plant” refers to a whole plant any part thereof, or a cell or tissue culture derived from a plant, comprising any of: whole plants, plant components, or organs (e.g., leaves, stems, roots, etc.), plant tissues, seeds, plant cells, and/or progeny of the same. A plant cell is a biological cell of a plant, taken from a plant or derived through culture from a cell taken from a plant.

“Promoter” as used herein refers to a nucleic acid sequence located upstream or 5′ to a translational start codon of an open reading frame (or protein-coding region) of a gene and that is involved in recognition and binding of RNA polymerase I, II, or III and other proteins (trans-acting transcription factors) to initiate transcription. A “plant promoter” is a native or non-native promoter that is functional in plant cells. Constitutive promoters are functional in most or all tissues of a plant throughout plant development. Tissue-, organ- or cell-specific promoters are expressed only or predominantly in a particular tissue, organ, or cell type, respectively. Rather than being expressed “specifically” in a given tissue, plant part, or cell type, a promoter may display “enhanced” expression, a higher level of expression, in one cell type, tissue, or plant part of the plant compared to other parts of the plant. Temporally regulated promoters are functional only or predominantly during certain periods of plant development or at certain times of day, as in the case of genes associated with circadian rhythm, for example. Inducible promoters selectively express an operably linked DNA sequence in response to the presence of an endogenous or exogenous stimulus, for example by chemical compounds (chemical inducers) or in response to environmental, hormonal, chemical, and/or developmental signals.

“Recombinant” in reference to a nucleic acid or polypeptide indicates that the material (for example, a recombinant nucleic acid, gene, polynucleotide, polypeptide, etc.) has been altered by human intervention. The term recombinant can also refer to an organism that harbors recombinant material, for example, a plant that comprises a recombinant nucleic acid is considered a recombinant plant.

“Transgenic plant” refers to a plant that comprises within its cells a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been altered by the presence of heterologous nucleic acid including those transgenic organisms or cells initially so altered, as well as those created by crosses or asexual propagation from the initial transgenic organism or cell. The term “transgenic” as used herein does not encompass the alteration of the genome (chromosomal or extrachromosomal) by conventional plant breeding methods (e.g., crosses) or by naturally occurring events such as random cross-fertilization, non-recombinant viral infection, non-recombinant bacterial transformation, non-recombinant transposition, or spontaneous mutation.

“Vector” is a polynucleotide or other molecule that transfers nucleic acids between cells. Vectors are often derived from plasmids, bacteriophages, or viruses and optionally comprise parts which mediate vector maintenance and enable its intended use. The term “expression vector” as used herein refers to a vector comprising operably linked polynucleotide sequences that facilitate expression of a coding sequence in a particular host organism (e.g., a bacterial expression vector or a plant expression vector).

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

### Example 1

### Constructs for Detecting and Optimizing Chromosomal Rearrangements Including Trans

Chromosomal Arm Exchange and Trans Fragment Targeting

A system for testing the efficiency of cis or trans chromosomal rearrangements in plant cells was designed. In several embodiments, the system employs chimeric reporter constructs, each comprising an N-terminal portion of a reporter coding sequence and a C-terminal portion of a reporter coding sequence that flank an intron. Intron sequences comprise at least one target site recognizable by a recombinase or endonuclease. Following recombination between chimeric reporter constructs at the target sites, the N-terminal and C-terminal portions of the reporter coding sequences each form an expression cassette capable of expressing the reporter coding sequence. Reporter coding sequences useful in these constructs encode reporters including fluorescent markers (e.g., GFP, YFP, BFP, CYP), enzymatic color markers (e.g., GUS), or herbicide tolerance selection markers (e.g., CP4).

In one embodiment, a first DNA molecule comprises the N-terminal portion of a first split reporter coding sequence linked to the C-terminal portion of a second split reporter coding sequence via a first intron. The intron comprises at least one target site recognizable by a genome editing reagent, such as a LoxP site or a target site for a CRISPR-associated protein/guide system. A second DNA molecule comprises the N-terminal portion of the second split reporter coding sequence linked to the C-terminal portion of the first split reporter coding sequence via a second intron, and the second intron also comprises at least one target site recognizable by a genome editing reagent, such as a LoxP site or a target site for a CRISPR-associated protein/guide system. Recombination results in the N-terminal and the C-terminal portions of the first reporter coding sequence being operably linked via the first intron, and the N-terminal and the C-terminal portions of the second reporter coding sequence being operably linked via the second intron. The resulting sequences are transcribed and processed to remove the introns, and at least one of the reporter coding sequences is expressed such that it can be detected.

In certain embodiments, sites of recombination such as native and synthetic LoxP and target sites for CRISPR-associated protein/guide systems, are comprised within introns to avoid potential frameshift as a result of error-prone non-homologous end joining (NHEJ). If small indels take place at a target site within the intron, correct splicing of the intron will take place and the reporters will still be expressed.

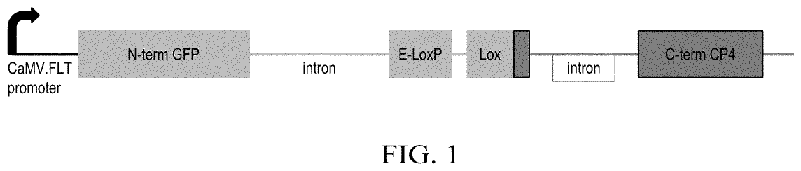

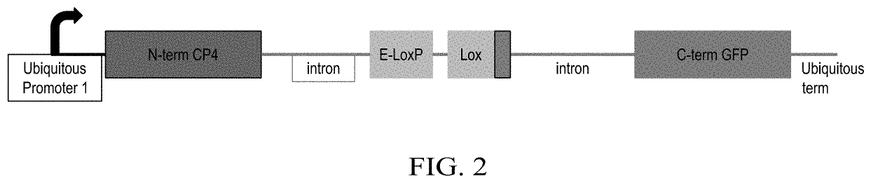

Exemplary constructs for testing the efficiency of cis and trans chromosomal exchanges in plant cells were designed as shown in FIGS. 1 and 2. FIG. 1 shows a first construct comprising a CaMV promoter, an N-terminal portion of a GFP coding sequence, a chimeric intron comprising at least one LoxP site, a target site for a CRISPR-associated protein/guide system, and a C-terminal portion of a CP4 coding sequence.

FIG. 2 shows a second construct for use in combination with the construct of FIG. 1 in a system for testing the efficiency of cis or trans chromosomal rearrangements. The second construct comprises a ubiquitous promoter 1, an N-terminal portion of the CP4 coding sequence, a chimeric intron comprising at least one LoxP site, a target site for a CRISPR-associated protein/guide system, and a C-terminal portion of the GFP coding sequence.

The constructs shown in FIGS. 1 and 2 can be used to detect recombination in a plant or plant cell by selecting for expression of GFP and CP4.

### Example 2

### Methods for Detecting and Optimizing Cis or Trans Chromosomal Exchanges

The split reporter system can be used with any gene editing system, for example with Cpf1/gRNA or Cas9/gRNA, and Cre/lox systems to study and optimize precision chromosome modification in plants. In particular, the system disclosed herein provides rapid and non-destructive assessment of cells for edited genomes, methods for the determining the frequency of chromosome rearrangements in cis and trans, and options for testing the efficiency of genome editing machinery driven by various promoters.

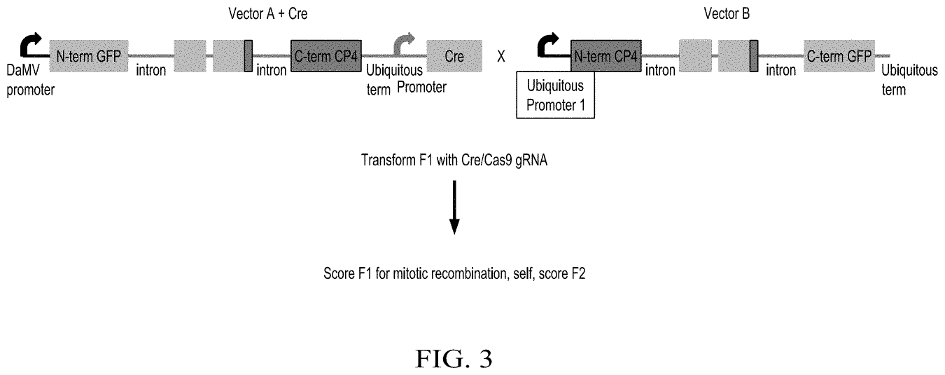

FIG. 3 shows a method for detecting and optimizing chromosomal rearrangement as described herein, using the constructs described in Example 1 and shown in FIGS. 1 and 2. Either or both of these constructs may be transformed into a plant using standard plant transformation methods. Transformation events containing Vector A or Vector B were produced, and transgene location in the genome was determined, for example using targeted sequencing methods. Libraries of Vector A and Vector B independent events were then used to study guided chromosomal rearrangement.

As shown in FIG. 3, plants comprising Vector A at a specific chromosomal location were crossed with plants comprising Vector B at a different chromosomal location. F1 plants from the cross were transformed with a sequence encoding a genome editing reagent, such as a recombinase or endonuclease, for example Cas9/gRNA, Cpf1/gRNA, or Cre. Recombination at a target site for the CRISPR-associated protein/guide system in the case of the Cas9/gRNA or Cpf1/gRNA system or LoxP site in the case of Cre, will produce expression of the GFP and CP4 markers. Expression of a reporter such as GFP, GUS, or CP4 can then be used to identify cis or trans chromosome exchanges.

In further embodiments, a sequence encoding a recombinase or endonuclease, such as Cas, Cpf1 or Cre, may be operably linked to one or both of the DNA constructs comprising the split reporter and target sequences under the control of a promoter. This method also eliminates a second transformation step to introduce Cre/Cas9 into cells or plants. Promoters with a desired pattern of expression may be used, for example the ubiquitous promoter 1, OsAct, AtEASE 35Smin, and AtDMC1.

A sequence encoding guide RNA (gRNA) may also be operably linked to one or both of the DNA constructs comprising the split reporter and target sequences under the control of a promoter. In certain embodiments, Vector A and Vector B comprise different target sites, and Vector A may further comprise a sequence encoding gRNA that recognizes the target site of Vector B, while Vector B may further comprise a sequence encoding gRNA that recognizes the target site of Vector A. Locating gRNA and its target site in different vectors, and therefore different parent plants, prevents an endonuclease from cutting the gRNA target site until and F1 progeny is created which comprises the Cas endonuclease, the target site, and its guide RNA.

### Example 3

### Design and Validation of Split Reporter Constructs in Corn Protoplasts

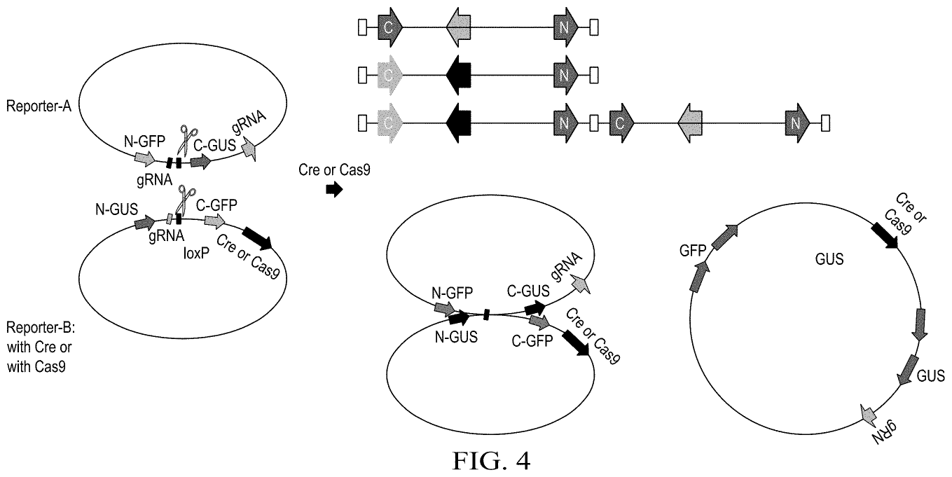

Methods of using split reporters for identification of cis or trans chromosomal exchange were tested and confirmed in isolated corn protoplasts. A schematic of plasmid recombination induced by expression of editing reagents (Cre or Cas9) is shown in FIG. 4. A double stranded break introduced by Cas9 or Cpf1 causes linearization of the plasmids followed by linkage at introns, expression, and splicing of repaired reporter mRNA. Expression of Cre causes recombination between two plasmids at the LoxP sites.

Split-reporter constructs were designed as shown in FIG. 4 to test recombination efficiency in corn protoplasts using components shown in Table 1. In one example, Reporter A comprised N-terminus GFP (SEQ ID NO: 1), gRNA (SEQ ID NO: 23), loxP (SEQ ID NO: 6), and C-GUS (SEQ ID NO: 4) sequences. Reporter A may further comprise promoter, intron, and terminator sequences disclosed herein or known in the art. Reporter B comprised N-GUS (SEQ ID NO:3), gRNA (SEQ ID NO: 23), loxP (SEQ ID NO: 6), and C-GFP (SEQ ID NO: 2) sequences. Reporter B may further comprise promoter, intron, and terminator sequences disclosed herein or known in the art. A Cre construct, for example comprising Cre_promoter (SEQ ID NO: 14), Cre_5′_intron (SEQ ID NO: 15), Cre coding sequence (SEQ ID NO: 13), and Cre_terminator (SEQ ID NO: 16), or a Cas construct, for example comprising a Cas_promoter (SEQ ID NO: 19), Cas_9_5′_intron (SEQ ID NO: 20), Cas9 coding sequence (SEQ ID NO: 17), and Cas9_terminator (SEQ ID NO: 18), may be included with Reporter A or B or transformed into plant comprising Reporter A or B. Assembly of reporter constructs using components disclosed herein or known in the art would be well within the capability of a person of skill in the art.

TABLE 1
Components for split-reporter constructs.

| SEQ ID NO | Component | Annotation |
|---|---|---|
| 1 | N-terminus GFP | GFP_S65T.nno |
| 2 | C-terminus GFP | GFP.nno |
| 3 | N-terminus GUS | uidA |
| 4 | C-terminus GUS | uidA |
| 5 | Tomato invertase gRNA | InvIh_Ts2 |
| 6 | LoxP site | lox1 |
| 7 | ReporterB_terminator | GT1 |
| 8 | ReporterB_5′_intron | Ubq1 |
| 9 | ReporterB_promoter | Ubq1 |
| 10 | ReporterA_terminator | Ccd |
| 11 | ReporterA_5'_intron | Act2 |
| 12 | ReporterA_promoter | FLT |
| 13 | Cre | Cre |
| 14 | Cre_promoter | Ubq1 |
| 15 | Cre_5′_intron | Ubq1 |
| 16 | Cre_terminator | Hsp17 |
| 17 | Cas9 | Sp.Cas9 13AA.zm 3′ |
| 18 | Cas9_terminator | LTP |
| 19 | Cas9_promoter | UbqM1 |
| 20 | Cas9_5′_intron | UbqM1 |
| 21 | gRNA Pol3 promoter | U6Chr8_Pol3 |
| 22 | sgRNA | sgRNA |

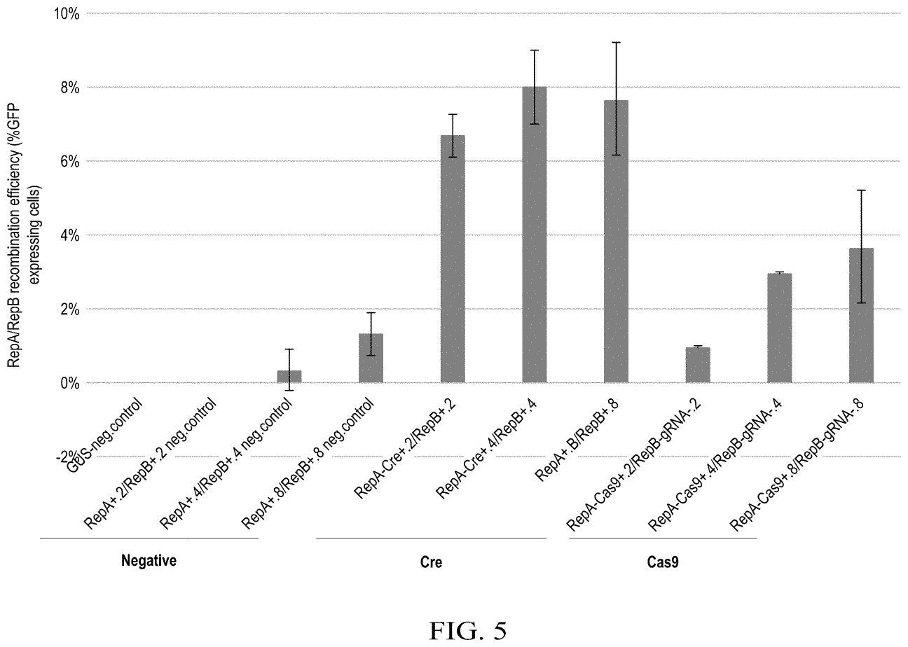

Recombination efficiency measured in corn protoplasts as a percent of cells expressing GFP is shown in FIG. 5. These protoplast assay results demonstrate recombination between Vector A and Vector B plasmids in the presence of Cre expression or maize codon-optimized Cas9 (SEQ ID NO: 17) in two different experiments. The recombination activity was detected by the number of GFP-expressing cells or percent of GFP-expressing cells which represents number or percent of cells in which recombination occurred. Recombination was plasmid concentration-dependent, and the highest levels of recombination were observed at concentrations of Vector A/Vector B of 0.4/0.4 pmole for Cre-driven recombination. The highest levels of recombination for Cas9-driven recombination were observed at concentrations of 0.8/0.8 pmole.

### Example 4

### Design and Validation of Cre Split Reporter Constructs in Soy Protoplasts

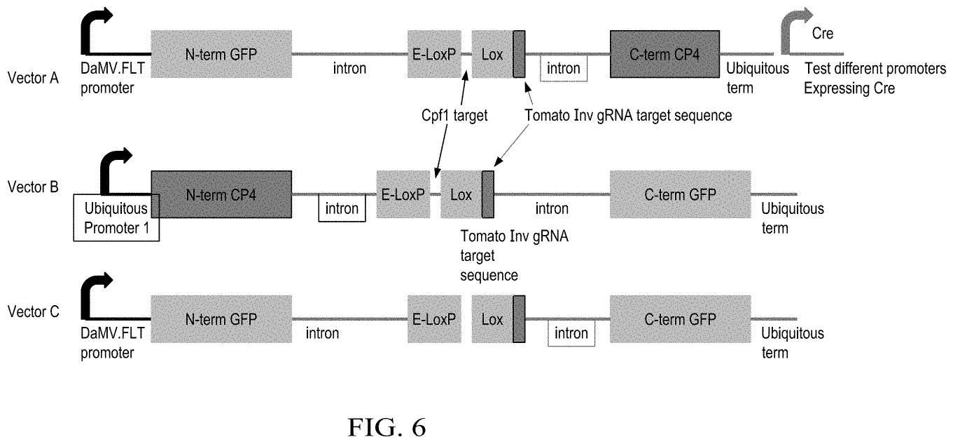

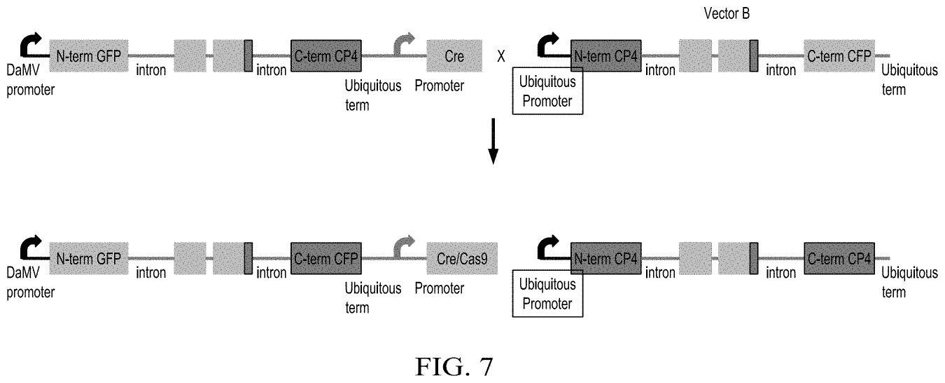

Vectors for a Cre split reporter system for determining recombination efficiency in soy cotyledon protoplasts are shown in FIG. 6. Vector A comprises a split reporter gene linked by an intron comprising Lox and gRNA sequences with or without a further Cre coding sequence driven by a separate promoter. Vector B comprises the intron, Lox, and gRNA sequences that are in Vector A. Vector C is a positive control. FIG. 7 shows the expected products of recombination in cells.

Split-reporter constructs were designed as shown in FIG. 6 to test recombination efficiency in soy protoplasts using components shown in Table 2. In one example, Reporter A comprised promoter (SEQ ID NO: 23), leader (SEQ ID NO: 24), N-term GFP (SEQ ID NO: 25), N-term LS1 intron (SEQ ID NO: 26), LoxP (SEQ ID NO: 27), gRNA target site (SEQ ID NO: 28), PAM site (SEQ ID NO: 29), C-term Act 7 intron (SEQ ID NO: 30), C-term CP4 (SEQ ID NO: 31), and terminator (SEQ ID NO: 32) sequences. Reporter A may further comprise promoter, intron, and terminator sequences disclosed herein or known in the art. Reporter B comprised promoter (SEQ ID NO: 33), leader (SEQ ID NO: 34), promoter intron (SEQ ID NO: 35), transit peptide (SEQ ID NO: 36), N-term CP4 (SEQ ID NO: 37), N-term intron (SEQ ID NO: 38), LoxP (SEQ ID NO: 39), gRNA target site (SEQ ID NO: 40), PAM site (SEQ ID NO: 41), C-term intron (SEQ ID NO: 42), C-term GFP (SEQ ID NO: 43), and terminator (SEQ ID NO: 45). Reporter B may further comprise promoter, intron, and terminator sequences disclosed herein or known in the art. A Cpf1 construct, for example comprising a promoter (SEQ ID NO: 45), one or more Cpf1 repeat non-coding RNAs (SEQ ID NO: 46), and a gRNA target site (SEQ ID NO: 47), may be included with Reporter A or B. Assembly of reporter constructs using components disclosed herein or known in the art would be well within the capability of a person of skill in the art.

TABLE 2
Exemplary components for split-reporter constructs.

| | | |
|---|---|---|
| SEQ ID NO | Description | Annotation |

VECTOR A ELEMENTS

| | | |
|---|---|---|
| 23 | Promoter | P-DaMV.FLT-1:1:13 |
| 24 | Leader sequence | L-DaMV.FLT:1 |
| 25 | N-term GFP | CR-Av.GFP_S65T.nno-1:4:3 |
| 26 | N-term LS1 intron | I-St.LS1:26 |
| 27 | Lox P | SP-P1.lox1:1 |
| 28 | gRNA target site | |
| 29 | PAM site | |
| 30 | C-term Act7 intron | I-At.Act7-1:1 |
| 31 | C-term CP4 | CR-AGRtu.aroA-CP4.nat:42 |
| 32 | Terminator | T-Mt.AC140914v20:1 |

VECTOR B ELEMENTS

| | | |
|---|---|---|
| 33 | Promoter | P-ubiquitous promoter 1 |
| 34 | Leader sequence | L-ubiquitous promoter 1 |
| 35 | Promoter intron sequence | I-ubiquitous promoter 1 |
| 36 | Transit peptide | TS-At.ShkG-CTP2:1 |
| 37 | N-term CP4 | I-ABTV.aaa:3 |
| 38 | N-term Intron | I-ABTV.aaa:2 |
| 39 | Lox P | SP-P1.lox1:1 |
| 40 | gRNA target site | NR-Gm.reporter_intron_1:1 |
| 41 | PAM site | |
| 42 | C-term Intron | I-St.LS1:27 |
| 43 | C-term GFP | CR-Av.GFP.nno-1:1:2 |
| 44 | Terminator | T-ubiquitous promoter 1 |
| 45 | Promoter | P-Gm.U6i:1 |
| 46 | Cpf1 repeat non-coding RNA | NR-LACba.Cpf1:2 |
| 47 | gRNA target site | NR-Gm.reporter_intron_1:1 |

A soy cotyledon assay was developed for assessing GFP expression as a measure of recombination efficiency in soy protoplasts. The seed coat was removed from 40 to 60 day old cotyledons, and tissue was sliced to 1 mm and subjected to plasmolysis for 1 hour at 26° C., digested for 2 hr at 26° C., and released for 5 min. Protoplasts were transferred to a 96-well plate and transformed via PEG-mediated transformation.

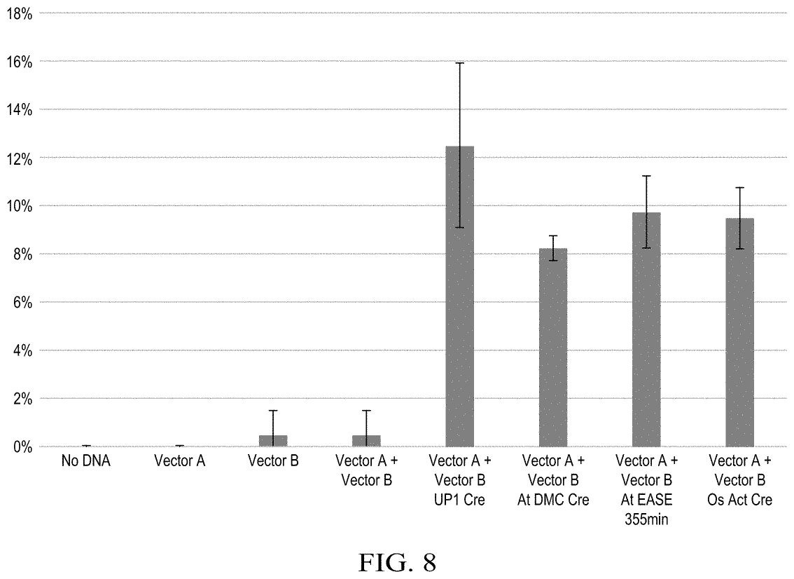

Vector A +/− Cre was co-transfected with Vector B into soy protoplasts. GFP expression that occurred through recombination of Vector A and Vector B at the Lox site was evaluated at 48 and 72 hours post transfection. FIG. 8 shows Operetta analysis of average percent GFP demonstrating that trans exchange was detected in soybean cotyledon protoplasts. These results validate the use of the Cre split reporter system in soy protoplasts, demonstrating that recombination occurred between Vector A +Cre and Vector B at the Lox site.

### Example 5

### Validation of Soy Cpf1 Split Reporter System in Soy Cotyledon Protoplasts

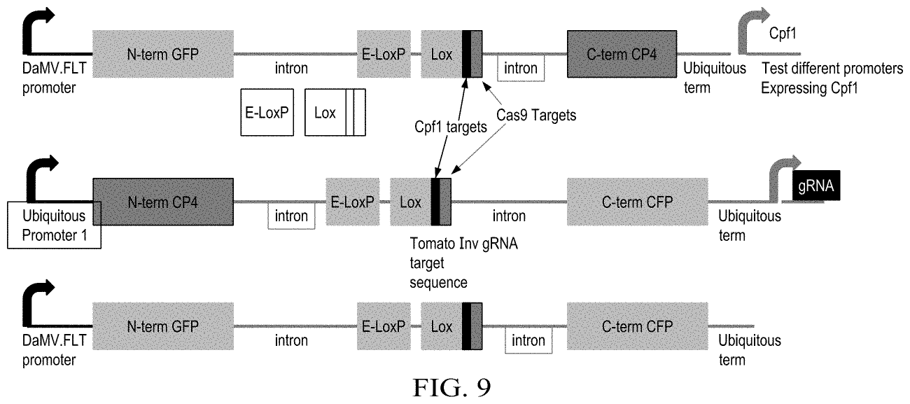

Vectors for a Cpf1 split reporter system for determining recombination efficiency in soy cotyledon protoplasts are shown in FIG. 9. Vector A comprises a split reporter gene linked by an intron comprising Lox and gRNA sequences with or without a further Cpf1 coding sequence driven by a separate promoter. Vector B comprises the intron, Lox, and gRNA sequences that are in Vector A. Vector C is a positive control.

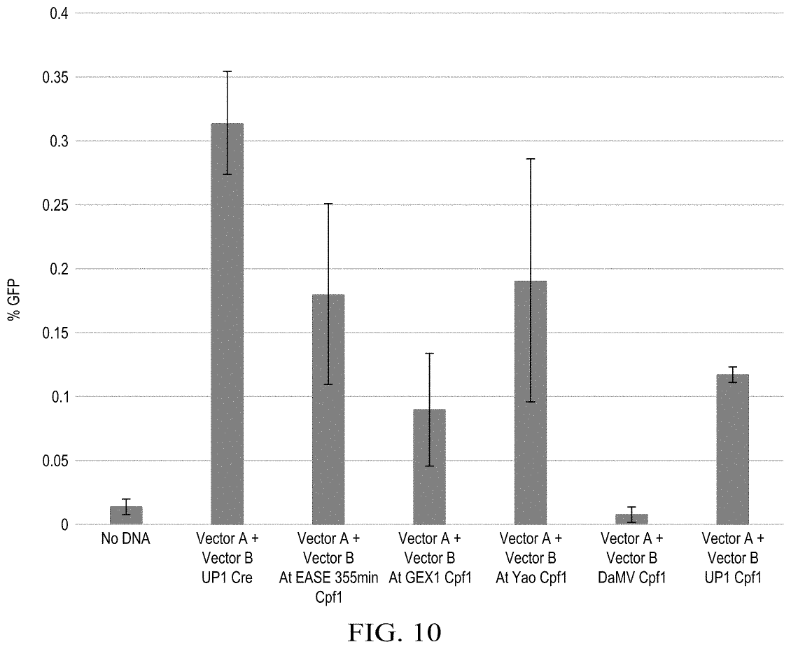

Vector A +/− Cpf1 was co-transfected with Vector B into soy protoplasts according to the assay described in Example 4. GFP expression that occurred through NHEJ of Vector A into Vector B was evaluated at 48 and 72 hours post transfection. FIG. 10 shows percent positive GFP cells and percent NHEJ. These results demonstrate the use of the Cfp1 split reporter system in soy protoplasts.

### Example 6

### Generation of Transformed Plants and Cells

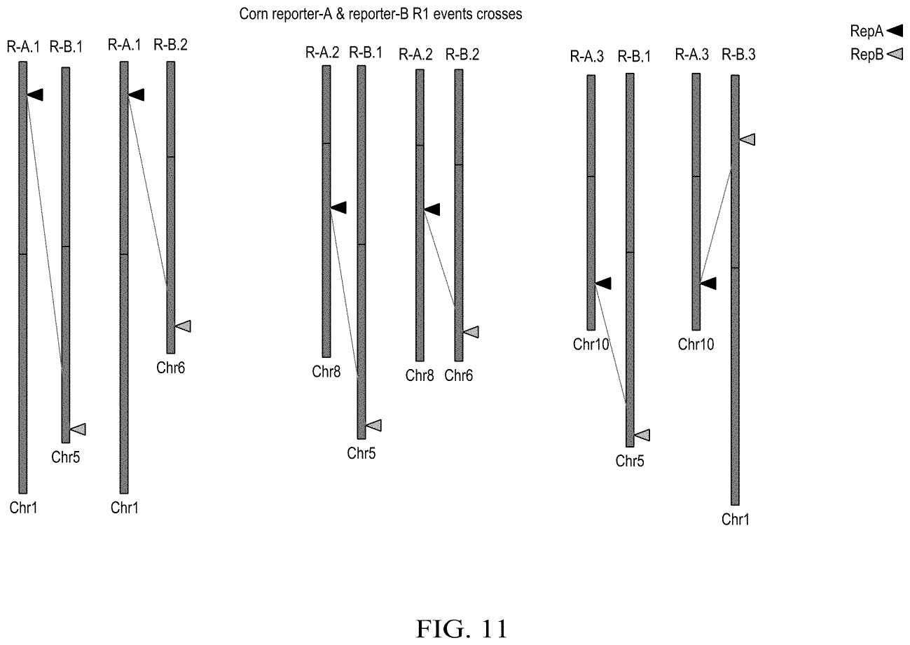

Constructs comprising a first split reporter and a second split reporter as shown in FIG. 4 (Reporter A and Reporter B) were transformed into corn plants. The transgene location in the corn genome was determined by targeted sequencing (SCIP). 7 events where random integration of Reporter A or Reporter B transgene into the genome is clearly defined were chosen for further testing. These events were self-crossed to produce R1 homozygous transgene events. The independent homozygous Reporter A and Reporter B events were crossed to produce a hemizygous population of F1 plants comprising both constructs as shown in FIG. 11. In addition, 3 out of 6 hemizygous for each reporter events were self-crossed to generate F2 generation where each transgene (Reporter A and Reporter B) are homozygous. These F1 and F2 materials will be harvested and evaluated for chromosomal rearrangement.

1. A pair of recombinant DNA molecules comprising:
a) a first DNA molecule comprising an N-terminal portion of a first reporter coding sequence and a C-terminal portion of a second reporter coding sequence that flank a first intron, wherein said first intron comprises a first target site recognizable by a first recombinase or endonuclease; and
b) second DNA molecule comprising an N-terminal portion of said second reporter coding sequence and a C-terminal portion of said first reporter coding sequence that flank a second intron, wherein said second intron comprises a second target site recognizable by a second recombinase or endonuclease;
wherein following recombination between said first and second DNA molecules at said target sites the N-terminal and C-terminal portions of said first reporter coding sequence form an expression cassette capable of expressing said first reporter coding sequence; and
wherein following recombination between said first and second DNA molecules at said target sites the N-terminal and C-terminal portions of said second reporter coding sequence form an expression cassette capable of expressing said second reporter coding sequence.

2. The pair of recombinant DNA molecules of claim 1, wherein said first and/or said second reporter coding sequence encodes a marker selected from the group consisting of a fluorescent marker, an enzymatic marker, and an herbicide tolerance selection marker.

3. (canceled)

4. The pair of recombinant DNA molecules of claim 1, wherein said first or said second recombinase is selected from the group consisting of a Cre recombinase, a FLP recombinase, and a TALE recombinase (TALER).

5. The pair of recombinant DNA molecules of claim 4, wherein said first or said second recombinase is a Cre recombinase, and said first or said second target site is a Lox site.

6. The pair of recombinant DNA molecules of claim 1, wherein said first or said second endonuclease is selected from the group consisting of a meganuclease, a Zinc Finger nuclease, a TALEN and a CRISPR-associated (Cas) endonuclease.

7. (canceled)

8. The pair of recombinant DNA molecules of claim 1, wherein said first DNA molecule further comprises a sequence encoding a Cas protein, and said second DNA molecule further comprises a sequence encoding a guide RNA.

9. The pair of recombinant DNA molecules of claim 8, wherein expression of said sequence encoding a recombinase or endonuclease is driven by a constitutive promoter, a tissue-specific promoter, or a meiotic promoter.

10. (canceled)

11. (canceled)

12. A cell comprising the pair of recombinant DNA molecules of claim 1.

13. A transgenic plant, plant seed or plant part comprising the pair of recombinant DNA molecules of claim 1.

14. A method for detecting cis or trans chromosomal rearrangement comprising:
a) obtaining a transgenic plant comprising a first DNA molecule comprising an N-terminal portion of a first reporter coding sequence and a C-terminal portion of a second reporter coding sequence that flank a first intron;
b) obtaining a transgenic plant comprising a second DNA molecule comprising an N-terminal portion of said second reporter coding sequence and a C-terminal portion of said first reporter coding sequence that flank a second intron;
c) crossing said first transgenic plant with said second transgenic plant to produce a progeny plant comprising said first DNA molecule and said second DNA molecule;
d) providing to at least a first cell of said progeny plant or a progeny thereof comprising said first DNA molecule and said second DNA molecule a recombinase or endonuclease that recognizes a target site in said first intron or a target site in said second intron; and
e) detecting recombination between said first and second DNA molecules at said target sites based on the expression of said first and second reporter coding sequences.

15. The method of claim 14, wherein said first DNA molecule further comprises a sequence encoding a Cas protein, and said second DNA molecule further comprises a sequence encoding a guide RNA.

16. (canceled)

17. The method of claim 14, wherein said first and/or said second reporter coding sequence encodes a marker selected from the group consisting of: a fluorescent marker, an enzymatic marker, and an herbicide tolerance selection marker.

18. The method of claim 17, wherein said first or said second reporter coding sequence encodes GFP, GUS, or CP4.

19. The method of claim 14, wherein said recombinase is selected from the group consisting of a Cre recombinase, a FLP recombinase, and a TALER.

20. The method of claim 14, wherein said endonuclease is selected from the group consisting of a meganuclease, a Zinc Finger nuclease, a TALEN and a Cas endonuclease.

21. (canceled)

22. A method for detecting a cis or trans chromosomal rearrangement comprising:
a) obtaining a transgenic plant comprising:
i) a first DNA molecule comprising an N-terminal portion of a first reporter coding sequence and a C-terminal portion of a second reporter coding sequence that flank a first intron, wherein said first intron comprises a first target site recognizable by a first recombinase or endonuclease; and
ii) a second DNA molecule comprising an N-terminal portion of said second reporter coding sequence and a C-terminal portion of said first reporter coding sequence that flank a second intron, wherein said second intron comprises a second target site recognizable by a second recombinase or endonuclease; and
wherein said first DNA molecule or said second DNA molecule further comprises a sequence encoding said first or said second recombinase or endonuclease;
b) detecting recombination between said first and second DNA molecules at said target sites based on the expression of said first and second reporter coding sequences.

23. The method of claim 22, wherein said first and/or said second reporter coding sequence encodes a marker selected from the group consisting of a fluorescent marker, an enzymatic marker, and an herbicide tolerance selection marker.

24. The method of claim 23, wherein said first or said second reporter coding sequence encodes GFP, GUS, or CP4.

25. The method of claim 22, wherein said first or said second recombinase is selected from the group consisting of a Cre recombinase, a FLP recombinase, and a TALER.

26. The method of claim 22, wherein said first or said second endonuclease is selected from the group consisting of a meganuclease, a Zinc Finger nuclease, a TALEN and a Cas endonuclease.

27. (canceled)


SEQUENCE LISTING (48 sequences)

SEQ ID NO: 1 - DNA, 379 bp, Unknown
  N-terminus GFP
atgggcaagggcgaggaactgttcactggcgtggtcccaatcctggtggaactggatggtgatgtgaacgggcacaagttctccgtcagcggagagggtgaaggtgatgccacctacggaaagctcaccctgaagttcatctgcactaccggaaagctccctgttccgtggccaaccctcgtcaccactttcacctacggtgttcagtgcttctcccggtacccagatcacatgaagcagcatgacttcttcaagagcgccatgcccgaaggctacgtgcaagaaaggactatctctttcaaggatgacgggaactacaagacacgtgccgaagtcaagttcgaaggtgataccctggtgaaccgcatcgagctgaaag

SEQ ID NO: 2 - DNA, 338 bp, Unknown
  C-terminus GFP
gtatcgatttcaaggaagatggaaacatcctcggacacaagctggagtacaactacaactcccacaacgtatacatcacggccgacaagcagaagaacggcatcaaggctaacttcaagatcaggcacaacatcgaagatggaagcgtgcaactggcggaccactaccagcagaacacgcccatcggcgatggccctgtcctgctgccggacaaccattacctgtccacgcaatctgccctctccaaggaccccaacgagaagagggaccacatggtcctgctggagttcgtgacggctgctgggatcacgcatggcatggatgaactctacaagtga

SEQ ID NO: 3 - DNA, 385 bp, Unknown
  N-terminus GUS
atggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtac

SEQ ID NO: 4 - DNA, 1427 bp, Unknown
  C-terminus GUS
gtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 5 - DNA, 20 bp, Unknown
  Tomato invertase gRNA
ggatctttccaagcttgagg

SEQ ID NO: 6 - DNA, 34 bp, Unknown
  LoxP site
ataacttcgtataatgtatgctatacgaagttat

SEQ ID NO: 7 - DNA, 232 bp, Unknown
  ReporterB_terminator
gttggcaatgcggataaagaataactaaataaataaataaataaattgcaagcaattgcgttgctgctatgtactgtaaaagtttcttataatatcagttctgaatgctaaggacatccctcaagatggtctttctatttttgtgttcccgttccaatgtactgttggtatcctcttggagattcatcaatatgagaaaacagagaatggacaaccctcccttatcttatgg

SEQ ID NO: 8 - DNA, 1044 bp, Unknown
  ReporterB_5′_intron
gtacggcgatcatcctccctttctctaccttctcttctctagactaggtcggcgatccatggttagggcctgctagttctgttcctgtttttccgtggctgcgaggtacaatagatctgatggcgttatgatggttaacttgtcatactcctgcggtgtgcggtctatagtgcttttaggacatcaatttgacctggctcgttcgagatcggcgatccatggttaggaccctaggcggtggagtcgggttagatccgcgctgtttgtgttagtagatggatgcgacctttacttcagacacgttctgattgttaacttgtcagcacctgggagtcctgggatggttctagctggttcgcagatgagatcgatttcatgatctgctgtatcttgtttcgttaggttccttttaatctatccgtggtattatgctaacctatgatatggttcgatcgtgctagctacgtcctgtgtcataatttttagcatgcccttttttgtttggttttgtctgattgggctgtagatcagagtatactgtttcaaactacctactggatatatttattaaatttgaatctgtatgtgtgtcacatatatcttcataattaaaatggatggaaagatatatggataggtacatgtgttgctgtgggttttactggtactttgttagatatacatgcttagatacatgaagcaacatgatgttacagttcaataattcttgtttacctaataaacaaataaggataggtgtatgttgctgtgggttttgctggtactttgttagatatatatgcttagatatatgaagcaacatcctgctacggtttaataattattgtttatatctaatagacaagcctgctttttaattattttgatatacttggatgatggcatacagcagctatgtgtggatttttaaatacccagcatcatgagcatgcatgaccctgccttagtatgctgtttatttgcttgagacttctttttttgttggtactcaccttttgtagtttggtgactcttctgcaggtg

SEQ ID NO: 9 - DNA, 1044 bp, Unknown
  ReporterB_promoter
ccaccggcaaacaaccacgaatttgtaatggtactaggcaaattctccgtttggcggtgtgtgccggccaattacacgtttttgcggtgtcctccgacaaaatttgccttttaaaaacaattttataagagaagctccggagataaaaggccgtcaatgttacaagagtgaagtcgtctactccctccatcccaaaaaatgtaattctaagtatgagttgtattattatttttggacaaaaggagtataccacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctcccc

SEQ ID NO: 10 - DNA, 376 bp, Unknown
  ReporterA_terminator
ttttattaaaatgcatgagcattatgatttgctctaatttgatcgctcatactatgtatgtgtgtgtatgtcgtcgttcgttgttgctctgcataatctgcatgcatgcatgggttaggttattacacataatctgcttgtgcatgcatgtgtagtatatgcatgtccttttctttttaaaccttttaattttctgatgagagtacgtgtgatcggtcgtctatcgatcatatatatatatgggtagcgagcttgtaccctcgcaaattgtacgttgttctctcttgtaatactactacatacatgcataaaaacatgtgtacgtttgtatactacgtgaaataaaagtttaaacaagctgtgttaaattatattg

SEQ ID NO: 11 - DNA, 499 bp, Unknown
  ReporterA_5′_intron
actccggtaacgatcttcgccactcattcaccctgtgtccctcgctagagtctccccttcctccataccgccgccccgcgcgggcgcggtacggtgctctcagatccgcccggcattgctcgctcgggccgatccgatctcgcatttgcgcgctcccgccgcggatccggccggatctcgcggaaatggggcgcgggtttctttggcatgctgtagatctgcttgtttcggctcgtgatctggaggcgccgcgggcgtttcggtaacgaaatgacctgatctgtggtggggttggggaatgccatttattagttcgttaattgctggttttctggtatcttattgggagggttcaattgatgcctttagagtagattttgtatgtagagcaaacaatggaaacaaatcatcgatagctttgcaatttgcctgctttgcttatttaattcctgcttgatttgacaaatgtggcatcgtgcgagctttcctgtaggtagaa

SEQ ID NO: 12 - DNA, 422 bp, Unknown
  ReporterA_promoter
atcaacggagaaacaaagataaaaatcaattactcacatgaaagagtattgatcacgagtcactatggagcgacaatctccagacaggatgtcagcatcttatcttcctttgaagaaagcatcatcaataacgatgtaatggtggggacatccactaagttattgctctgcaaacagctcaaaaagctactggccgacaatcataattgctcggcatgtgcaggtggggcctccactagcaataatacaagctttacagcttgcagtgactcatcctccaataatggagaaaaagacgtcagcagtgacgaacaagggtcgaaagacttgcctatataagggcattctcccctcagttgaagatcatcgaaagttggagcaataaactctctcttcaacaaatctatcttttatcttttatc

SEQ ID NO: 13 - DNA, 1221 bp, Unknown
  Cre
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 14 - DNA, 807 bp, Unknown
  Cre_promoter
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaag

SEQ ID NO: 15 - DNA, 1006 bp, Unknown
  Cre_5′_intron
gtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacaggtg

SEQ ID NO: 16 - DNA, 210 bp, Unknown
  Cre_terminator
ctgcatgcgtttggacgtatgctcattcaggttggagccaatttggttgatgtgtgtgcgagttcttgcgagtctgatgagacatctctgtattgtgtttctttccccagtgttttctgtacttgtgtaatcggctaatcgccaacagattcggcgatgaataaatgagaaataaattgttctgattttgagtgcaaaaaaaaaggaatt

SEQ ID NO: 17 - DNA, 4353 bp, Unknown
  Cas9
atgggatctaagaagagaagaattaaacaagatatggacaagaagtactccataggcctcgacatcgggaccaatagcgtgggttgggcggtgatcacagatgattacaaggtgccatccaagaagtttaaggtgctcggtaatacagacaggcattccataaagaaaaacctcattggcgcacttctattcgggtccggggagacggcggaggcgacgcgtttgaaacgaacggcgcgcaggcgctacacgcggaggaagaacaggatctgctacctgcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggagatctttagcaacgagatggcaaaggtcgacgattcatttttccaccgcctggaagaatcttttctcgttgaggaggacaaaaaacacgagcgtcatccaatcttcggtaacattgttgacgaggtggcataccatgagaaatacccgacaatctaccatctgcggaagaagctggctgactcgaccgataaggctgatcttaggttgatctacctcgccctggcgcacatgatcaagttcagggggcactttctcattgagggagatctaaacccagataacagcgatgtggataagctgttcatccaactcgtgcagatctacaaccagcttttcgaagagaaccctatcaacgcaagccgtgttgacgcaaaagccatattgagtgcaaggctctctaaatcccgccggctcgaaaatctgatagcccaacttcccggagaaaaaaggaatggattatttggcaacctcatcgctttatccctaggactaacaccgaattttaagtccaattttgatctcgcagaggatgctaaattgcaactctcaaaagacacatacgatgatgacctggacaatctgcttgcacagatcggtgatcaatatgcggatctcttcctcgcggctaaaaacctgtctgacgccatacttctgagtgacatattgagagtgaacagcgaaataactaaagcacccctgtctgcaagcatgatcaagcgctacgacgaacaccaccaagaccttactctcctgaaggccctcgtgaggcaacagctgcctgagaagtacaaggagattttctttgatcagtcgaagaacgggtatgctgggtacatcgatgggggtgctagccaggaggaattctacaagttcatcaagcctattctggaaaaaatggatggtactgaagagctattggtgaaattgaatagagaagatcttttacgtaagcaacgaactttcgacaatgggagtatcccacatcagatacatctgggtgaactccacgcaatactccggcgccaggaagacttttacccatttcttaaagataaccgcgagaaaatagagaagatccttacattcagaatcccttactatgtcgggccactggctcggggcaacagtcggttcgcatggatgacacgcaaatcagaggaaaccataacaccttggaactttgaagaggtggtggataagggtgcatcagcccaatctttcatcgagagaatgacaaactttgataagaatttgcccaacgaaaaagtccttccaaaacatagtcttttgtatgaatatttcacggtctataatgaattaacaaaagtaaaatacgtgacagagggaatgaggaagcctgcgtttctgtccggcgagcaaaagaaagctatcgtcgacttgttgttcaagaccaaccggaaggtgactgttaaacaattgaaagaggactattttaaaaagatcgagtgctttgattcagttgagattagcggagtggaagatcggtttaatgcctcattgggcgcttaccacgatctcctgaagattattaaggataaggattttttggataatgaggagaacgaggatattctcgaggatattgttcttacacttacactatttgaagaccgcgggatgatcgaggaaagactgaagacttacgctcatctcttcgatgacaaggttatgaagcagctgaagcggaggcgctataccggttggggtaggctcagccgcaaactgattaacggcatcagggataagcagtccggtaagacaatcctggattttctgaagtccgatggatttgcgaacagaaattttatgcagctgattcacgacgactcattaacttttaaagaagacattcagaaagcacaggtttcaggccagggtcacagtctgcatgagcagatcgctaatttggcaggctcaccagctatcaagaagggcattcttcaaacagtgaagatagtagatgaactcgtaaaggtaatgggacataagcctgaaaatattgttattgaaatggcgagagaaaaccaaaccacacagaagggtcagaagaactcaagagaacggatgaaaaggatagaagaggggataaaggagctgggttctcagatccttaaagaacatccagtggagaacacgcaattacaaaacgagaagctgtatctttactaccttcaaaatggtagagatatgtacgtggatcaggaattggacattaatcgtctgtctgattacgatgtggaccatatagttcctcagtcatttatcaaggatgattcaatcgacaataaggtcctaactcgttcagacaagaatagaggtaaatcggacaatgtgccaagcgaggaagtggtgaagaaaatgaagaattactggcggcagttacttaacgcaaagcttattacccaacggaagttcgataacctcacgaaagctgaacgcggtggtttatctgaactggataaggctggatttatcaagcgccaactggtggagactcgacagattacgaaacatgtggcacagatcttagatagtagaatgaacacaaagtatgatgaaaatgacaagcttatccgggaggtcaaagtgatcaccctgaagtcaaagttggtttccgattttcggaaggattttcagttctataaagtccgagaaataaacaattatcatcacgcgcatgatgcttaccttaacgcggttgtagggaccgctcttatcaaaaaatacccaaagctggaatcggagttcgtgtatggggactataaggtgtatgatgttcgtaaaatgattgcaaaaagtgaacaagagatcggtaaagcaactgcgaagtattttttctacagtaacattatgaacttttttaagactgagataacgctggcaaacggtgaaattcgcaaacgtcctctgatagaaacgaatggtgagacaggggagatagtatgggataaggggcgtgactttgctaccgtacgaaaggtgctctctatgccacaggtcaatatcgtcaagaagacagaggtccagactggaggtttttctaaggaatcgatactcccaaagagaaattcagacaagctgattgcacgtaagaaagattgggacccgaaaaagtacggaggctttgattccccaacagttgcctattctgtgcttgttgtcgcaaaggtggagaagggtaaatcaaagaagctgaagtcggttaaagaactactcggcatcacaatcatggagaggagcagcttcgagaagaacccgatagacttcctcgaggccaagggctataaagaggtcaagaaagacttaataattaagctcccaaagtactctctatttgaactcgagaacggccgcaagaggatgctcgctagcgctggtgaacttcagaagggcaacgaacttgccctcccctccaagtatgttaattttctttatttggcaagccattacgagaagttaaaaggctcgcctgaagataatgagcaaaaacagctatttgtcgaacagcataagcattacctagatgaaattattgagcagatctccgagtttagcaaaagagtgatccttgccgacgcaaatctcgacaaagttctcagtgcctacaacaagcacagggacaagcctatacgggagcaggctgaaaacatcatccatctgtttaccctcactaaccttggagcccctgccgccttcaaatactttgacactacaattgatcgcaagaggtataccagcacaaaggaggttctagacgcgacacttattcatcagagcattacaggactatacgaaacgcgtatcgacttgtcccaacttgggggagacggatctaagaagagaagaattaaacaagat

SEQ ID NO: 18 - DNA, 300 bp, Unknown
  Cas9_terminator
attaatcgatcctccgatcccttaattaccataccattacaccatgcatcaatatccatatatatataaaccctttcgcacgtacttatactatgttttgtcatacatatatatgtgtcgaacgatcgatctatcactgatatgatatgattgatccatcagcctgatctctgtatcttgttatttgtataccgtcaaataaaagtttcttccacttgtgttaataattagctactctcatctcatgaaccctatatataactagtttaatttgctgtcaattgaacatgatgatcgatg

SEQ ID NO: 19 - DNA, 955 bp, Unknown
  Cas9_promoter
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaag

SEQ ID NO: 20 - DNA, 1053 bp, Unknown
  Cas9_5′_intron
gtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtc

SEQ ID NO: 21 - DNA, 200 bp, Unknown
  gRNA Pol3 promoter
cggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaatt

SEQ ID NO: 22 - DNA, 83 bp, Unknown
  sgRNA
gttttagagctagaaatagcaagttaaaataaggctagtccgttatcaacttgaaaaagtggcaccgagtcggtgcttttttt

SEQ ID NO: 23 - DNA, 365 bp, Unknown
  Promoter
atcaacggagaaacaaagataaaaatcaattactcacatgaaagagtattgatcacgagtcactatggagcgacaatctccagacaggatgtcagcatcttatcttcctttgaagaaagcatcatcaataacgatgtaatggtggggacatccactaagttattgctctgcaaacagctcaaaaagctactggccgacaatcataattgctcggcatgtgcaggtggggcctccactagcaataatacaagctttacagcttgcagtgactcatcctccaataatggagaaaaagacgtcagcagtgacgaacaagggtcgaaagacttgcctatataagggcattctcccctcagttgaagatc

SEQ ID NO: 24 - DNA, 57 bp, Unknown
  Leader sequence
atcgaaagttggagcaataaactctctcttcaacaaatctatcttttatcttttatc

SEQ ID NO: 25 - DNA, 379 bp, Unknown; identical to SEQ ID NO: 1
  N-term GFP

SEQ ID NO: 26 - DNA, 68 bp, Unknown
  N-term LS1 intron
gtaagtttctgcttctacctttgatatatatataataattatcattaattagtagtaatataatattt

SEQ ID NO: 27 - DNA, 35 bp, Unknown
  Lox P
ataacttcgtatagcatacattatacgaagttata

SEQ ID NO: 28 - DNA, 23 bp, Unknown
  gRNA target site
caagcttgaggaggctataactt

SEQ ID NO: 29 - DNA, 4 bp, Unknown
  PAM site
tttc

SEQ ID NO: 30 - DNA, 274 bp, Unknown
  C-term Act7 intron
aagttgtttagttcgaaatgacttcgtgtatgctcgattgatctggttttaatcttcgatctgttaggtgttgatgtttacaagtgaattctagtgttttctcgttgagatctgtgaagtttgaacctagttttctcaataatcaacatatgaagcgatgtttgagtttcaataaacgctgctaatcttcgaaactaagttgtgatctgattcgtgtttacttcatgagcttatccaattcatttcggtttcattttactttttttttagtgaa

SEQ ID NO: 31 - DNA, 680 bp, Unknown
  C-term CP4
gccgcggcaagctcaccggccaagtcatcgacgtgccgggcgacccgtcctcgacggccttcccgctggttgcggccctgcttgttccgggctccgacgtcaccatcctcaacgtgctgatgaaccccacccgcaccggcctcatcctgacgctgcaggaaatgggcgccgacatcgaagtcatcaacccgcgccttgccggcggcgaagacgtggcggacctgcgcgttcgctcctccacgctgaagggcgtcacggtgccggaagaccgcgcgccttcgatgatcgacgaatatccgattctcgctgtcgccgccgccttcgcggaaggggcgaccgtgatgaacggtctggaagaactccgcgtcaaggaaagcgaccgcctctcggccgtcgccaatggcctcaagctcaatggcgtggattgcgatgagggcgagacgtcgctcgtcgtgcgtggccgccctgacggcaaggggctcggcaacgcctcgggcgccgccgtcgccacccatctcgatcaccgcatcgccatgagcttcctcgtcatgggcctcgtgtcggaaaaccctgtcacggtggacgatgccacgatgatcgccacgagcttcccggagttcatggacctgatggccgggctgggcgcgaagatcgaactctccgatacgaaggctgcctga

SEQ ID NO: 32 - DNA, 500 bp, Unknown
  Terminator
aagggctctctgtcatgatttcatactttcattattgagctctgtaattacaattatgaccatgagaacatctcttattgtgtggccttttaattgctgatgttagtactgaaccaaagcttatcgtgatgatgtaaaagcaataagtacttgtttgtagcttctttgtgtctccctttgggcttaatacatctgtttagtgttgtggctttggcatagacttctcttggtaataatgccttgcaatgcaaaatttcaattatcaaattctattatgttctcaccttatggtaacagcttaccctgtggaagatgagattcttgagttgagtcattgccaatttttggcattagcttttgaattagtgaattttgacaaaaattaccgtgacactgattttgttgaagctcttaagtgtagtttttacaaaatttcagtggctcgttgtgattatgtcaaactcacggcgaatgtagttcttacagaatttcagtggctc

SEQ ID NO: 33 - DNA, 1199 bp, Unknown
  Promoter
gacaacaacatgcttctcatcaacatggagggaagagggagggagaaagtgtcgcctggtcacctccattgtcacactagccactggccagctctcccacaccaccaatgccaggggcgagctttagcacagccaccgcttcacctccaccaccgcactaccctagcttcgcccaacagccaccgtcaacgcctcctctccgtcaacataagagagagagagaagaggagagtagccatgtggggaggaggaatagtacatggggcctaccgtttggcaagttattttgggttgccaagttaggccaataaggggagggatttggccatccggttggaaaggttattggggtagtatctttttactagaattgtcaaaaaaaaatagtttgagagccatttggagaggatgttgcctgttagaggtgctcttaggacatcaaattccataaaaacatcagaaaaattctctcgatgaagatttataaccactaaaactgccctcaattcgaagggagttcaaaacaattaaaatcatgttcgaattgagtttcaatttcactttaacccctttgaaatctcaatggtaaaacatcaacccgtcaggtagcatggttctttttattcctttcaaaaagagttaattacaaacagaatcaaaactaacagttaggcccaaggcccatccgagcaaacaatagatcatgggccaggcctgccaccaccctccccctcctggctcccgctcttgaatttcaaaatccaaaaatatcggcacgactggccgccgacggagcgggcggaaaatgacggaacaacccctcgaattctaccccaactacgcccaccaacccacacgccactgacaatccggtcccacccttgtgggcccacctacaagcgagacgtcagtcgctcgcagcaaccagtgggcccacctcccagtgagcggcgggtagatctggactcttacccacccacactaaacaaaacggcatgaatattttgcactaaaaccctcagaaaaattccgatattccaaaccagtacagttcctgaccgttggaggagccaaagtggagcggagtgtaaaattgggaaacttaatcgagggggttaaacgcaaaaacgccgaggcgcctcccgctctatagaaaggggaggagtgggaggtggaaaccctaccacaccgcagagaaa

SEQ ID NO: 34 - DNA, 86 bp, Unknown
  Leader sequence
ggcgtcttcgtactcgcctctctccgcgccctcctccgccgccgctcgccgccgttcgtctccgccgccaccggctagccatccag

SEQ ID NO: 35 - DNA, 896 bp, Unknown
  Promoter intron sequence
gtaaaacaaacaaaaacggatctgatgcttccattcctccgtttctcgtagtagcgcgcttcgatctgtgggtggatctgggtgatcctggggtgtggttcgttctgtttgatagatctgtcggtggatctggccttctgtggttgtcgatgtccggatctgcgttttgatcagtggtagttcgtggatctggcgaaatgttttggatctggcagtgagacgctaagaatcgggaaatgatgcaatattaggggggtttcggatggggatccactgaattagtctgtctccctgctgataatctgttcctttttggtagatctggttagtgtatgtttgtttcggatagatctgatcaatgcttgtttgttttttcaaattttctacctaggttgtataggaatggcatgcggatctggttggattgccatgatccgtgctgaaatgcccctttggttgatggatcttgatattttactgctgttcacctagatttgtactcccgtttatacttaatttgttgcttattatgaatagatctgtaacttaggcacatgtatggacggagtatgtggatctgtagtatgtacattgctgcgagctaagaactatttcagagcaagcacagaaaaaaatatttagacagattgggcaactatttgatggtctttggtatcatgctttgtagtgctcgtttctgcgtagtaatcttttgatctgatctgaagataggtgctattatattcttaaaggtcattagaacgctatctgaaaggctgtattatgtggattggttcacctgtgactccctgttcgtcttgtcttgataaatcctgtgataaaaaaaattcttaaggcgtaatttgttgaaatcttgttttgtcctatgcagcctg

SEQ ID NO: 36 - DNA, 228 bp, Unknown
  Transit peptide
atggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgc

SEQ ID NO: 37 - DNA, 688 bp, Unknown
  N-term CP4
atgcttcacggtgcaagcagccggcccgcaaccgcccgcaaatcctctggcctttccggaaccgtccgcattcccggcgacaagtcgatctcccaccggtccttcatgttcggcggtctcgcgagcggtgaaacgcgcatcaccggccttctggaaggcgaggacgtcatcaatacgggcaaggccatgcaggcgatgggcgcccgcatccgtaaggaaggcgacacctggatcatcgatggcgtcggcaatggcggcctcctggcgcctgaggcgccgctcgatttcggcaatgccgccacgggctgccgcctgacgatgggcctcgtcggggtctacgatttcgacagcaccttcatcggcgacgcctcgctcacaaagcgcccgatgggccgcgtgttgaacccgctgcgcgaaatgggcgtgcaggtgaaatcggaagacggtgaccgtcttcccgttaccttgcgcgggccgaagacgccgacgccgatcacctaccgcgtgccgatggcctccgcacaggtgaagtccgccgtgctgctcgccggcctcaacacgcccggcatcacgacggtcatcgagccgatcatgacgcgcgatcatacggaaaagatgctgcagggctttggcgccaaccttaccgtcgagacggatgcggacggcgtgcgcaccatccgcctggaag

SEQ ID NO: 38 - DNA, 270 bp, Unknown
  N-term Intron
gtgagtctctagatccgttcgcttgattttgctgctcgttagtcgttattgttgattctctatgccgatttcgctagatctgtttagcatgcgttgtggttttatgagaaaatctttgttttgggggttgcttgttatgtgattcgatccgtgcttgttggatcgatctgagctaattcttaaggtttatgtgttagatctatggagtttgaggattcttctcgcttctgtcgatctctcgctgttatttttgtttttttcagtgaagtg

SEQ ID NO: 39 - DNA, 35 bp, Unknown; identical to SEQ ID NO: 27
  Lox P

SEQ ID NO: 40 - DNA, 23 bp, Unknown
  gRNA target site
caagcttgaggaggctataactt

SEQ ID NO: 41 - DNA, 4 bp, Unknown
  PAM site
tttc

SEQ ID NO: 42 - DNA, 99 bp, Unknown
  C-term Intron
agaatgtagtatatagcaattgcttttctgtagtttataagtgtgtatattttaatttataacttttctaatatatgaccaaaatttgttgatgtgcag

SEQ ID NO: 43 - DNA, 338 bp, Unknown; identical to SEQ ID NO: 2
  C-term GFP

SEQ ID NO: 44 - DNA, 582 bp, Unknown
  Terminator
cagggttcttgcctggtgccttggcaatgcttgattactgctgctatcctatgatctgtccgtgtgggcttctatctatcagtttgtgtgtctggttttgaaaaacatttgcttttcgattatgtagggtttgcttgtagctttcgctgctgtgacctgtgttgtttatgtgaaccttctttgtggcatctttaatatccaagttcgtggtttgtcgtaaaacgaagcctctacttcgtaaagttgtgtctatagcattgaaatcgtttttttgctcgagaataattgtgacctttagttggcgtgaaactagttttggatatctgattctctggttcgcaatcttgagatcgtcgctgcttaggtgagctaagtgatgttcctaagtaaatgctcctcaccagaatacgtagctgtgtgaaaagagaacgcgtgaatacgtagctgtgtaaagattgtgtcccaagtaaacctcagtgatttttgtttggatttttaatttagaaacattcgactgggagcggctagagccacacccaagttcctaactatgataaagttgctctgtaacagaaaacacca

SEQ ID NO: 45 - DNA, 427 bp, Unknown
  Promoter
cgataaaaatgttttaaacgatatatattataaaaaaaaacgtttcaaaaataaatacaaaaatgtttttaaatatatataatttaactcattaaagaaaataaaaatgcaagtgcggtgacaagacaagctaaaagttgcaaaagaaatggcagggctataaggctcacctactcctggatttaccaaattttggttcgtccctatactcgaaaaataaaacaaaataaatttcagtatcttcgtttttgtatgctttgactgtgaggcgaggccaactttcttcttctgtctgagatgaattttgtttgcctcctgtgaaggatgtatcattcaaagtgaatgttttgcaactgccagtagtcccacatcgaccaaatattcttattacagtgtgtttatatagcacctggagaaggaatgggtt

SEQ ID NO: 46 - DNA, 20 bp, Unknown
  Cpf1 repeat non-coding RNA
aatttctactaagtgtagat

SEQ ID NO: 47 - DNA, 23 bp, Unknown
  gRNA target site
caagcttgaggaggctataactt

SEQ ID NO: 48 - DNA, 20 bp, Unknown
  Cpf1 repeat non-coding RNA
aatttctactaagtgtagat